(19) United States
(12) Patent Application Publication
(10) Pub. No.: US 2016/0106927 A1
(43) Pub. Date: Apr. 21, 2016

(54) Drug Delivery Device and System with Pre-Set Dose Feature

(71) Applicant: Novo Nordisk A/S, Bagsværd (DK)

(72) Inventors: Mads Moeller, Hundested (DK); Michael Svendsmark Hansen, Frederiksberg C (DK)

(73) Assignee: Novo Nordisk A/S, Bagsvaerd (DK)

(21) Appl. No.: 14/893,334

(22) PCT Filed: May 26, 2014
(86) PCT No.: PCT/EP2014/060840

§ 371 Date: Nov. 23, 2015

(30) Foreign Application Priority Data
May 27, 2013 (EP) 13169352.5

(51) Int. Cl. A61M 5/315; A61M 5/20

(52) U.S. Cl. CPC A61M 5/31535; A61M 5/20; A61M 2205/52

(57) ABSTRACT

A drug delivery device comprises drug expelling means and a drug-filled cartridge, the cartridge comprising an outlet and an axially displaceable piston. The drug expelling means comprises a drive member adapted to engage and axially move the piston to thereby expel an amount of drug from the cartridge through the outlet, and a motor for moving the drive member. The device further comprises a display adapted to display a dose amount to be expelled, communication means allowing dose setting data to be received from an external device, and an electronic controller adapted to store received dose setting data, control the display, and control the motor to move the drive member to thereby expel an amount of drug corresponding to a set dose.

The present invention relates motor driven drug delivery devices and systems. Especially, the invention addresses the issue of setting a dose amount for a drug to be expelled.

### BACKGROUND OF THE INVENTION

In the disclosure of the present invention reference is mostly made to the treatment of diabetes by delivery of insulin, however, this is only an exemplary use of the present invention.

Drug delivery devices have greatly improved the lives of patients who must self-administer drugs and biological agents. Drug delivery devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230 and US 2011/306927. Performing the necessary insulin injection at the right time and in the right size is essential for managing diabetes, i.e. compliance with the specified insulin regimen is important.

Having regard to the above, it is an object of the present invention to provide devices, systerns and methods which allow a user to set and/or administer a given dose of drug in a convenient and safe way.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a drug delivery device is provided comprising drug expelling means and a drug-filled cartridge or means for receiving a drug-filled cartridge, the cartridge comprising an outlet and an axially displaceable piston. The drug expelling means comprises a drive member adapted to engage and axially move the piston to thereby expel an amount of drug from the cartridge through the outlet, and a motor for moving the drive member. The device further comprises communication means allowing dose setting data to be received from an external device, and an electronic controller adapted to store received dose setting data, and control the motor to move the drive member to thereby expel an amount of drug corresponding to a set dose. By this arrangement a given amount of drug to be expelled can be set and stored in the drug delivery device by external means, this providing a drug delivery device which is both simple and easy to use when a dose corresponding to a stored value is to be delivered.

The controller may comprise a clock and be adapted to store dose setting data representing at least two pre-set doses, each pre-set dose being associated with a time period of the day and/or week, the controller being adapted to control the motor to move the drive member to thereby expel a stored dose amount according to the actual time.

In an exemplary embodiment the drug delivery device comprises a display adapted to display a dose amount to be expelled, the electronic controller being adapted to control the display.

The drug delivery device may be provided with first switch means for actuating the device between an idle condition and an activated condition, e.g. an on/off switch, and second switch means for actuating the controller to expel an amount of drug corresponding to a set dose, wherein the controller is adapted to display a stored dose amount when the device is actuated from the idle to the activated condition, e.g. when a protective cap is removed, the stored dose amount representing a pre-set dose. In this way the device is ready to expel the displayed pre-set dose without the user to having to set the dose for each new dosing event.

The drug delivery device may be provided with a clock and being adapted to store dose setting data representing at least two pre-set doses, each pre-set dose being associated with a time period of the day and/or week, wherein the device is adapted to display a stored dose amount according to the actual time when the device is actuated from the idle to the activated condition. For example, two pre-set doses may be stored corresponding to a morning time period respectively an evening time period. The time period may have any desired length, e.g. 12, 8 or 4 hours. If the device is turned on outside a time period a message or simply a dose of zero may be displayed.

The display may be adapted to display period information representing a period of the day, each pre-set dose being associated with period information. For example, a morning dose amount may be accompanied by a morning symbol, e.g. a rising sun, and an evening dose amount may be accompanied by an evening symbol, e.g. a moon or star.

The drug delivery device may further comprise user actuated dose setting means allowing a user to adjust a pre-set dose to be expelled or to set a desired dose if no pre-set is stored or displayed.

In a further aspect of the invention a drug delivery device is provided in combination with an external dose setting device, the dose setting device comprising a display adapted to show a dose to be set, the dose representing a pre-set dose, means for setting the dose corresponding to a desired dose amount, and means for transmitting dose setting data to the drug delivery device. In this way simple and safe programming of one or more pre-set doses can be performed.

A given set dose may be associated with period information, e.g. representing a morning or an evening dose as described above. A plurality of doses may be set and transmitted, each dose being associated with period information. For example, two doses as described above may be transmitted with corresponding morning and evening period information.

In an exemplary embodiment the setting device is adapted to display two or more doses to be set as well as corresponding period information simultaneously. For example, when it is desired to program a morning and an evening pre-set dose the display may initially show two values each representing a dose amount and each being associated with period information, e.g. symbols as described above. The initially shown values may each be zero, e.g. when the programming feature is used for the first time, or the values may represent stored values representing presently programmed dose sizes. Using the setting means the user may now set/adjust each of the simultaneously shown doses. When done the dose data is transmitted to the drug delivery device by wire or wireless communication.

The setting device may be a stationary device as a PC or a portable device as a smartphone, each being provided with application software providing the described functionality. The latter may comprise a touch screen allowing for easy setting or adjusting of the displayed dose amounts.

In a yet further aspect of the invention a method of setting a dose of drug to be expelled from a drug delivery device is provided. The method comprises the steps of (i) providing a drug delivery device adapted to receive dose setting data from an external device, the drug delivery device comprising an electronic controller adapted to store received dose setting data and control a motor to move a drive member to thereby expel an amount of drug corresponding to a set dose, and an external dose setting device comprising a display adapted to show a dose to be set, the dose representing a pre-set dose, means for setting the dose corresponding to a desired dose amount, and means for transmitting dose setting data to the drug delivery device, (ii) setting a dose on the external dose setting device, and (iii) transmitting data representing the set dose from the external dose setting device to the drug delivery device.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of the exemplary embodiments reference will be made to the use of insulin.

### BRIEF DESCRIPTION OF THE DRAWINGS

In the following the invention will be further described with reference to the drawings, wherein

FIG. 1 shows a pen-formed drug delivery device,

FIG. 2 shows a smartphone set up to set and transmit a single pre-set dose value,

FIG. 3 shows a smartphone set up to set and transmit one, two or three pre-set dose values corresponding to a daily regimen,

FIG. 4 shows a pen-formed drug delivery device in combination with a smartphone set up to set and transmit two pre-set dose values corresponding to a twice daily regimen,

FIG. 5 shows a first embodiment of a drug delivery device platform, and

FIGS. 6 and 7 show a second embodiment of a drug delivery device platform.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

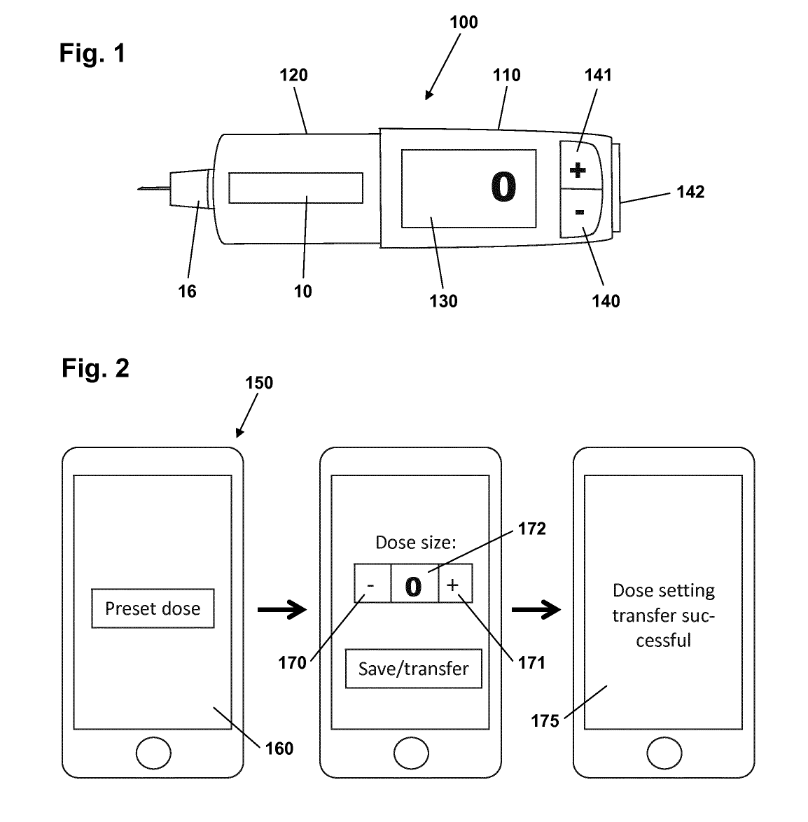

FIG. 1 shows schematically an embodiment of a pen-formed drug delivery device 100 adapted to receive a disposable pre-filled drug cartridge, the device comprising a body portion 110 with an electronically controlled motor driven expelling mechanism, a cartridge holder portion 120 adapted to receive and hold a drug filled cartridge 10, means allowing a needle assembly 16 to be mounted in fluid communication with the cartridge (may be formed as part of the disposable cartridge), a display 130 adapted to show a set dose to be expelled, up-down setting keys 140, 141 allowing a user to set or adjust a desired dose, and actuation means 142 to actuate the expelling mechanism to expel an amount of fluid drug from the cartridge corresponding to the set dose size, e.g. units of insulin (IU). In the shown embodiment the display is adapted to show text, however, alternatively the display may be a simple 7-segment display which could be adapted to display one or more additional symbols. A cap (not shown) is provided to cover the cartridge portion and a needle if attached. The cap may used to turn on the device. A more detailed description of a drug delivery device suitable as a platform for embodiments of the present invention will be given below with reference to FIGS. 4-7.

FIG. 2 shows an external dose setting device in the form of a generic smartphone 150, the smartphone comprising a touch display 160 serving to both display information and allow input of data. FIG. 2 shows a simple set-up in which the smartphone is provides with application software, e.g. a downloaded “app”, allowing a single dose of drug to be set and transmitted wirelessly, e.g. by NFC or Bluetooth, to a corresponding drug delivery device, e.g. of the type shown in FIG. 1. In the shown embodiment the dose is set by utilizing up-down “buttons”, the actual dose size being shown in number of IU, this corresponding to the user interface of the shown delivery device 100. Alternatively the dose size may be set using a “spinning wheel” design as utilized to set values on many smartphones. When a desired dose size has been set it can be transmitted to the delivery device which in response may transmit an acknowledgement 175. Before transmitting dose data for the first time the smartphone and the pen may have been paired to each other to allow for safe transfer of data only. The initial pairing may also be used to set the clock of the delivery device. If no device is identified the set data will be saved and can be transferred later.

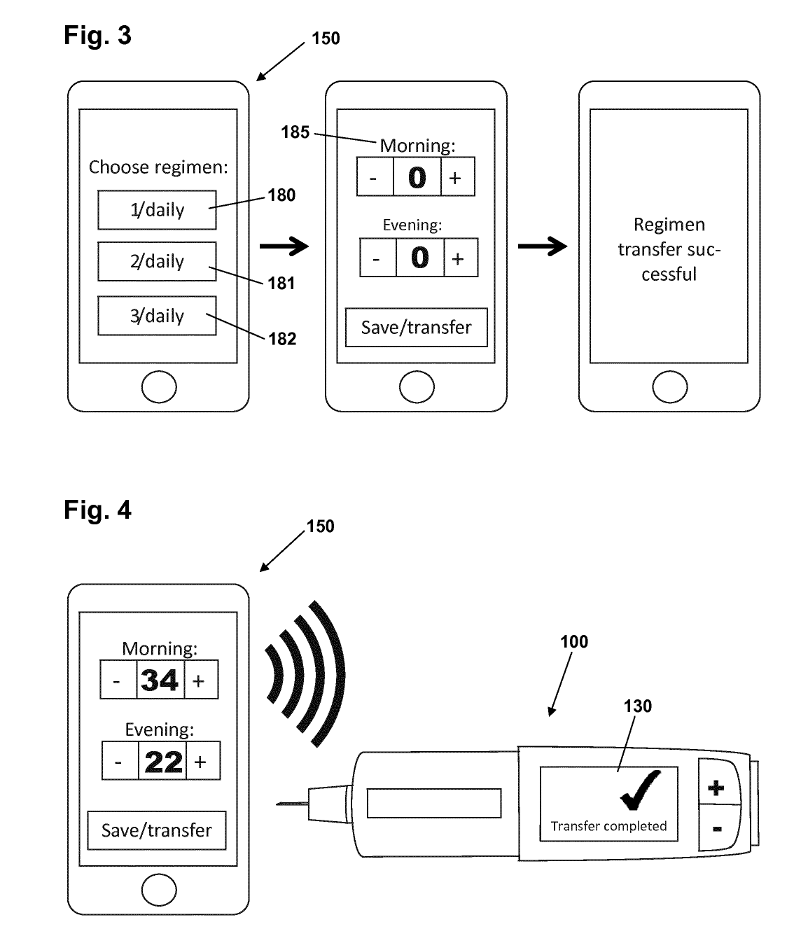

FIG. 3 shows a set-up in which the smartphone is provided with application software allowing a regimen comprising up to three daily injections to be programmed, each alternative being represented by a button. After the user has chosen the number of daily injections, the display will show all values to be set in a single display view, the values being shown in combination with a period identifier 185, here “morning” and “evening”. Using the touch screen the user can then simply set or adjust the doses to the desired pre-set sizes, the combined showing providing a full overview of the regimen without the need to toggle back and forth between different display showings.

FIG. 4 shows a specific example in which the smartphone 150 has been used to set-up a twice daily regimen comprising a morning dose of insulin of 34 IU and an evening dose of 22 IU. When the delivery device 100 has received the full regimen data set, safe transmission of data is indicated in the display 130. The delivery device is now set up for easy daily use.

When the user takes off the cap the display will show a pre-set dose amount corresponding to the actual time of the day. The time period may have any desired length, e.g. 12, 8 or 4 hours. For example, a morning dose may be shown and indicated when the cap is removed between 6:00 and 10:00, and an evening dose may be shown and indicated when the cap is removed between 18:00 and 24:00. If the device is turned on outside these time periods a message or simply a dose of zero may be displayed, which dose can then be set manually. Further, a given pre-set dose can also be adjusted if needed.

The expelled amounts may be stored in a log which may be displayed on the device or transferred to an external device for convenience of use, e.g. the above-mentioned smartphone.

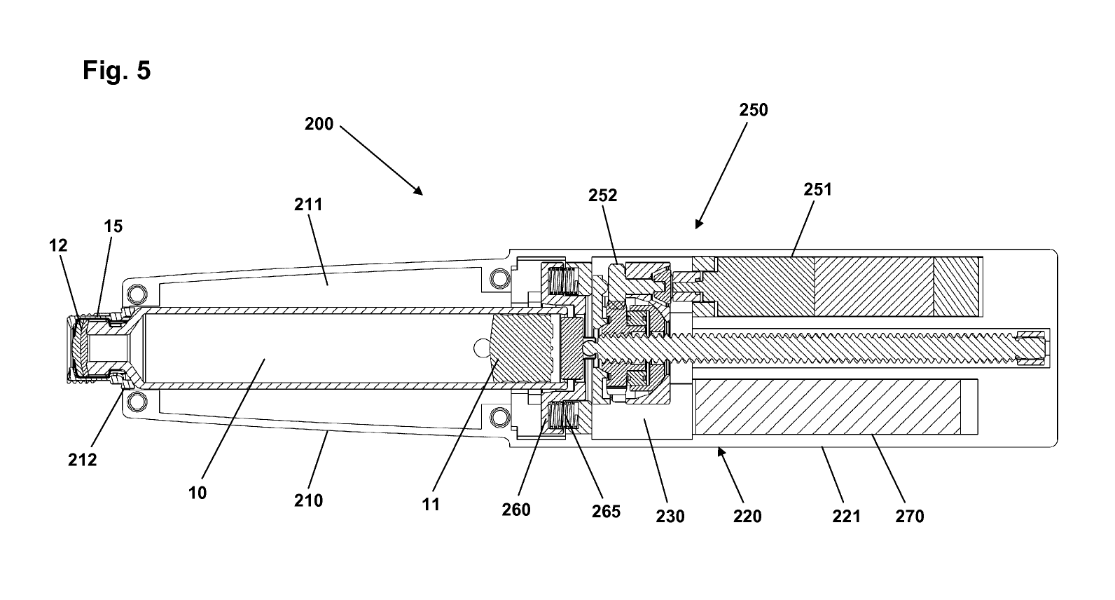

Turning to FIG. 5 a first embodiment 200 of a drug delivery device suitable as a platform for embodiments of the present invention will be described. More specifically, the device comprises a cap part (not shown) and a main part having a proximal body or drive assembly portion 220 with a housing 221 in which a motorized drug expelling assembly 250, electronic controller and communication circuitry 270 and an electric power source are arranged, and a distal cartridge holder portion 210 with a compartment 211 in which a drug-filled cartridge 10 is arranged and retained in place. The cartridge comprises a generally cylindrical main portion with an axially displaceable piston 11 and a distal outlet portion 12 comprising a needle-penetrable septum. The cartridge is further provided with distal coupling means in the form of a needle hub mount 15 having, in the shown example, an external thread adapted to engage an inner thread of a corresponding hub of a needle assembly. The cartridge may for example contain an insulin, a GLP-1 or a growth hormone formulation. The device further comprises dose setting means allowing a user to set a dose of drug to be expelled as well as a display for showing the set dose, e.g. as shown in FIG. 1.

In the shown embodiment the device is designed to be loaded by the user with a new cartridge through a distal receiving opening 212 in the cartridge holder assembly, the cartridge holder comprising closure means (not shown) operatable by a user between an open position in which a cartridge can be inserted respectively removed, and a closed position in which an inserted cartridge is held in place. The closure means may be of the same type as described with respect to FIG. 5 below. In order to axially position the cartridge, the device comprises a seat member 260 adapted to receive the proximal end of the cartridge, the seat member being biased in the proximal direction by springs 265 thereby forcing the cartridge into contact with the closure means.

When incorporating an embodiment of the present invention in the platform device of FIG. 5, the necessary controller and communications functionality could cost-effectively be integrated in the electronic controller circuitry 270.

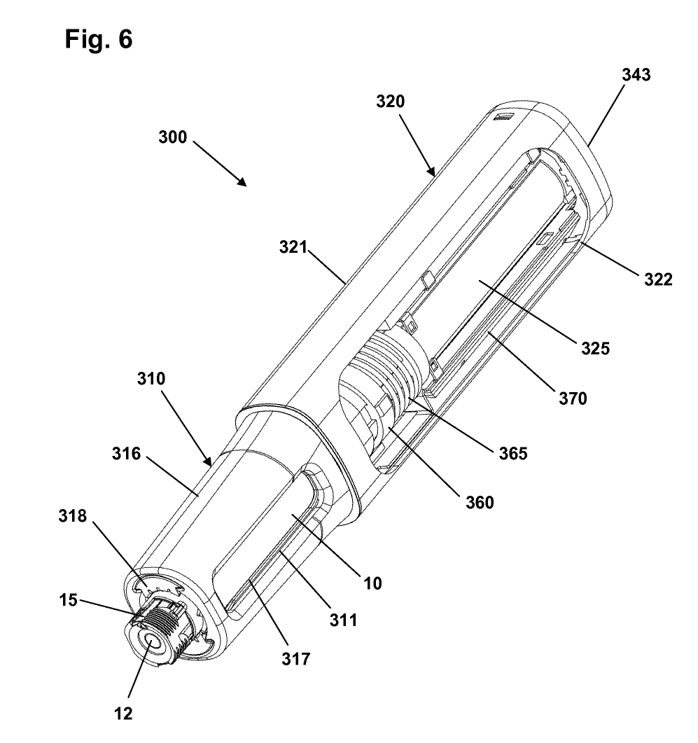

Turning to FIG. 6 a second embodiment 300 of a drug delivery device suitable as a platform for embodiments of the present invention will be described. More specifically, the device comprises a cap part (not shown) and a main part having a proximal body or drive assembly portion with a housing 321 in which a drug expelling mechanism and associated control and communication electronics are arranged, and a distal cartridge holder assembly 310 forming a compartment in which a drug-filled transparent cartridge 10 can be arranged and retained in place, the cartridge holder assembly comprising a pair of opposed inspection openings 311. The housing comprises an opening 322 adapted to receive a display frame member (not shown) in which a LCD as well as user input keys are mounted, e.g. as shown in FIG. 1. With the frame member removed, it can be seen that the device comprises a generally tubular chassis member 325, in which a generally cylindrical expelling assembly is mounted (see below). The device further comprises a control and communication assembly 370, a bias assembly comprising a bias member 360 and a spring 365, and a proximal release button 343.

A pair of dose setting input keys (not shown) serves to manually set a desired dose of drug shown in the LCD and which can then be expelled when the release button 90 is actuated. The device is designed to be loaded by the user with a new cartridge through a distal receiving opening in the cartridge holder assembly.

The cartridge 10 comprises a cylindrical body portion, a distal outlet portion 12 with a distal needle-penetrable septum, and an axially displaceable piston having a proximal surface allowing a piston driver forming part of the expelling mechanism (see below) to engage the piston. The cartridge may for example contain an insulin, a GLP-1 or a growth hormone formulation. The cartridge is provided with distal coupling means in the form of a needle hub mount 15 having, in the shown example, combined thread and bayonet coupling means, each being adapted to engage an inner thread or bayonet coupling means of a corresponding hub of a needle assembly. The shown exemplary hub mount further comprises a circumferential flange with a number of distally facing pointed projections serving as a coupling means for the cartridge holder assembly as will be described in more detail below. A hub mount of the shown type is described in U.S. Pat. No. 5,693,027. Alternatively the needle hub mount may be formed as part of the cartridge holder, e.g. in the form of a “split” hub mount having two parts arranged on each side of the gripping shoulders.

As shown, the cartridge holder assembly 310 has the same general appearance as a traditional cartridge holder which is detachably coupled to the housing by e.g. a threaded coupling or a bayonet coupling and into which a new cartridge can be received as well as removed through a proximal opening, i.e. it comprises no additional user operated release or locking means. Instead, what appears merely to be the cartridge holder per se is in fact user operated coupling means in the form of an outer rotatable tubular actuation sleeve 316 operated by the user to control movement of cartridge holding means in the form of an inner cartridge holder member 317 to thereby open and close gripping shoulders 318 configured to grip and hold a cartridge. More specifically, each gripping shoulder is provided with a plurality of gripping teeth spaced circumferentially to provide a plurality of gaps, each tooth having a triangular configuration with a proximally oriented pointed end, thereby creating a plurality of gaps having a distally oriented pointed configuration, this allowing the above-described distally facing pointed projections on the cartridge to be received between the teeth to thereby serve as a gripping means when the cartridge holding means has been moved into engagement with the cartridge. In this way an easy-to-use front loaded drug delivery device is provided which appears as a traditional rear loaded device and which is also actuated by rotational movement to mount and remove a cartridge, the resemblance providing for ease of acceptance and adaptation among users accustomed to traditional types of rear loaded drug delivery devices.

When it is time to mount a new cartridge the outer tube member 316 is rotated e.g. 90 degrees by which action the gripping shoulders 318 are moved distally and slightly outwards, this allowing the mounted cartridge to be removed. For ease of operation the cartridge may be moved distally a certain distance as the shoulders are moved, e.g. by engagement with arms forming the gripping shoulders and/or by additional spring means providing a biasing distally directed force (see below). Depending on the design of the locking and actuation mechanism the gripping shoulders may be able to be left in the open position or they may be retracted automatically as the outer tube member is rotated backwards by return spring means. Whether or not a spring is provided the cartridge holder may be provided with locking means allowing the outer tube member to be securely parked in either the open or closed position, e.g. by a rotational snap lock. When a new cartridge is inserted the drive expelling means has to be in a state allowing a new cartridge with a proximally positioned piston to be inserted. An exemplary embodiment providing this functionality will be described below.

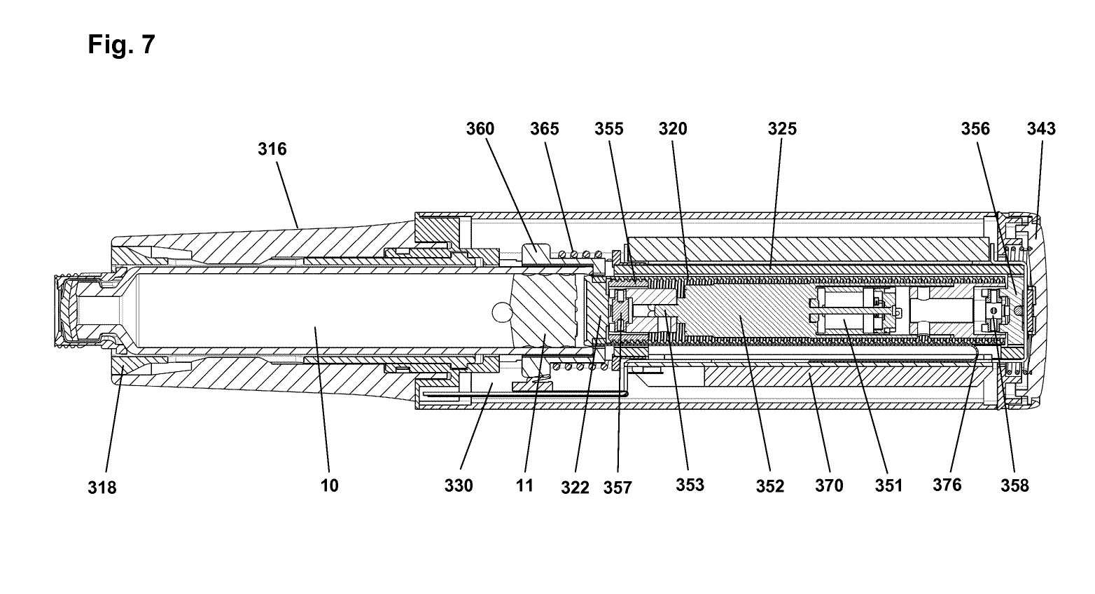

Turning to FIG. 7 a cross-sectional view of the drug delivery device 300 of FIG. 6 is shown with a mounted cartridge 10 and with the piston tube 320 (see below) in a fully retracted position. More specifically, the actuation sleeve 316 has been rotated to its operational position and the cartridge holder gripping shoulders 318 have been retracted to their closed position thereby retracting the cartridge to its fully inserted position, thereby also moving the bias member 360 proximally against the bias of the spring 365. In the shown embodiment a cartridge switch 375 is hereby being actuated, this providing a signal to the device controller that two actions can be assumed to have taken place: (i) a cartridge has been inserted and (ii) the cartridge holder has been closed, this initiating that the drive head is moved distally into contact with the cartridge piston. In the shown embodiment it is contemplated that detection of contact between the drive head and the piston is detected by electronic sensor means arranged in the drive head, e.g. using proximity detection as disclosed in WO 2013/144152.

FIG. 7 also shows the expelling assembly in greater detail. More specifically, the expelling assembly is in the form of a motor-in-piston assembly comprising an interior motor and gearbox drive assembly mounted axially and rotationally locked to the proximal end of the chassis, and an outer axially displaceable piston tube 320 with a distal drive head 322 adapted to engage the piston 11 of a loaded cartridge, the piston tube comprising a number of guide projections adapted to non-rotationally engage corresponding guide means of the chassis.

The motor-gear drive assembly comprises a tubular main portion 310 composed of a proximal motor assembly 351 and a distal gearbox assembly 352 having a rotatable drive shaft 353 defining a z-axis of rotation. The assembly further comprises a distal cylindrical drive member 355 having an outer thread adapted to be arranged in engagement with the piston drive tube inner thread. At the proximal end a disc-formed chassis connector 356 is arranged. In the shown embodiment the drive assembly is provided with flexible joints in the form of a distal universal joint 357 arranged between the drive shaft and the drive member and a proximal universal joint 358 arranged between the motor assembly proximal portion and the chassis tube proximal portion. A corresponding drive assembly is described in greater detail in patent application EP 14166859.0 which is hereby incorporated by reference.

A number of further details can be seen in FIG. 7. The release button 343 is received in the housings proximal opening with a spring providing a proximally directed biasing force on the button. A flexible ribbon 376 with a plurality of conductors is arranged with a U-bend between the electronics portion 370 and the sensors (not shown) arranged in the piston head, this allowing the piston tube and piston head to travel axially with the U-bend moving correspondingly.

When incorporating an embodiment of the present invention in the platform device of FIGS. 6 and 7, the necessary controller and communications functionality could cost-effectively be integrated in the control assembly 370.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device comprising:
a drug-filled cartridge or means for receiving a drug-filled cartridge (10), the cartridge comprising an outlet and an axially displaceable piston,
drug expelling structure comprising:
a drive member adapted to engage and axially move the piston to thereby expel an amount of drug from the cartridge through the outlet, and
a motor for moving the drive member,
communication structure allowing dose setting data to be received from an external device, and
an electronic controller adapted to:
store received dose setting data, and
control the motor to move the drive member to thereby expel an amount of drug corresponding to a set dose.

2. A drug delivery device as in claim 1,
wherein the controller comprises a clock and is adapted to store dose setting data representing at least two pre-set doses, each pre-set dose being associated with a time period of the day and/or week, and
wherein the controller is adapted to control the motor to move the drive member to thereby expel a stored dose amount according to the actual time.

3. A drug delivery device as in claim 1, further comprising a display adapted to display a dose amount to be expelled, the electronic controller being adapted to control the display.

4. A drug delivery device as in claim 3, further comprising:
first switch structure for actuating the device between an idle condition and an activated condition,
second switch structure for actuating the controller to expel an amount of drug corresponding to a set dose,
wherein the controller is adapted to display a stored dose amount when the device is actuated from the idle to the activated condition, the stored dose amount representing a pre-set dose.

5. A drug delivery device as in claim 3,
wherein the controller comprises a clock and is adapted to store dose setting data representing at least two pre-set doses, each pre-set dose being associated with a time period of the day and/or week, and
wherein the controller is adapted to display a stored dose amount according to the actual time when the device is actuated from the idle to the activated condition.

6. A drug delivery device as in claim 3, wherein the display is adapted to display period information representing a period of the day, each pre-set dose being associated with period information.

7. A drug delivery device as in claim 3, further comprising a user actuated dose setting structure allowing a user to adjust a pre-set dose to be expelled.

8. A drug delivery device as in claim 3, in combination with an external dose setting device, the dose setting device comprising:
a display adapted to show a dose to be set, the dose representing a pre-set dose,
a structure for setting the dose corresponding to a desired dose amount, and
a structure for transmitting dose setting data to the drug delivery device.

9. The combination of claim 8, wherein a given set dose is associated with period information.

10. The combination of claim 9, wherein a plurality of doses can be set and transmitted, each dose being associated with period information.

11. The combination of claim 10, wherein the setting device display is adapted to display simultaneously at least two doses to be set and thereto corresponding period information.

12. A method of setting a dose of drug to be expelled from a drug delivery device, comprising:
providing a drug delivery device adapted to receive dose setting data from an external device, the drug delivery device comprising an electronic controller adapted to store received dose setting data and control a motor to move a drive member to thereby expel an amount of drug corresponding to a set dose, and
an external dose setting device comprising a display adapted to show a dose to be set, the dose representing a pre-set dose, a structure for setting the dose corresponding to a desired dose amount, and a structure for transmitting dose setting data to the drug delivery device,
setting a dose on the external dose setting device, and
transmitting data representing the set dose from the external dose setting device to the drug delivery device.
